(19) United States
(12) Patent Application Publication
(10) Pub. No.: US 2007/0225210 A1
(43) Pub. Date: Sep. 27, 2007

(54) Therapeutic vaccine compositions for the treatment of type 1 diabetes

(76) Inventor: Peter Blackburn, New York, NY (US)

Correspondence Address: Thomas Hoxie, Esq.;Hoxie & Tso LLP, Suite 300E, 374 Millburn Avenue, Millburn, NJ, 07041, US

(21) Appl. No.: 11/293,585

(22) Filed: Dec. 2, 2005

Related U.S. Application Data
(63) Continuation-in-part of application No. PCT/US04/17247, filed on Jun. 1, 2004.
(60) Provisional application No. 60/474,857, filed on Jun. 2, 2003.

(51) Int. Cl. A61K 38/28; C07K 14/62

(52) U.S. Cl. 514/3; 530/303

(57) ABSTRACT

The invention concerns therapeutic vaccine compositions that comprise modified Insulin B chain components suitable for use as immunogenic agents for treatment and prevention of Type 1 Diabetes.

### CROSS-REFERENCE TO RELATED APPLICATION

This application is a continuation-in-part of International Application PCT/US2004/017247 filed Jun. 1, 2004, designating the United States, which claims priority to U.S. provisional patent application Ser. No. 60/474,857, filed Jun. 2, 2003, the disclosures of which related applications are incorporated herein in their entirety.

### FIELD OF THE INVENTION

The invention concerns therapeutic vaccine compositions that comprise modified Insulin B chain components suitable for use as immunogenic agent for treatment and prevention of Type 1 Diabetes.

### BACKGROUND OF THE INVENTION

Type 1 diabetes is an autoimmune disease affecting the pancreatic islet cells. Antibodies to various islet cell proteins are found in the sera of type 1 diabetics early in the progression of their disease. These include antibodies towards: Insulin, Vardi, et al. (1988) Diabetes Care 11, 736-739); glutamic acid decarboxylase (GAD65), Hagoplan et al. (1993) Diabetes 42: 631-636. Evidence suggests that repeat administration of low doses of insulin B chain to individuals with, or at risk of, type 1 diabetes can prevent or reduce the symptoms of diabetes and the insulin-dependency of the patient, Keller, et al. (1993) Lancet 341: 927-928. Immunization with insulin B chain, (see U.S. Pat. No. 5,891,435 and US Patent Application 2003/0045467) or fragments of insulin B chain, in particular residues 9-23 (see U.S. Pat. No. 5,594,100; Daniel, et al. (1996) PNAS 93: 956-960), have been demonstrated to protect from the onset of diabetes in NOD mice.

### SUMMARY OF THE INVENTION

The invention relates to immunogenic compositions comprising an insulin B chain analog peptide, wherein at least one of the two cysteine residues in the insulin B chain is substituted by a serine, a threonine or alanine residue and the use of such insulin B chain analogs to treat diabetes and pre-diabetic conditions. Certain embodiments of the invention concern immunogenic compositions comprising an insulin B chain analog peptide comprising a single cysteine residue wherein the analogue is dimerized via a homodimeric sulfide linkage through the sulfhydryl groups of a cysteine residue. In some embodiments, the insulin B chain analog peptide may be conjugated to an immunogenic carrier, wherein at least one of the two cysteine residues in the insulin B chain is substituted by a serine, a threonine or alanine residue. In addition, the carboxy terminal threonine residue may be replaced with serine or alanine. Methods of treating diabetes or a pre-diabetic condition in a human subject which comprise administering to the subject the immunogenic compositions comprising the modified insulin B chain analogs are also provided.

### DETAILED DESCRIPTION OF THE INVENTION

The amino acid sequence for the A and B-chains of insulin are as follows: 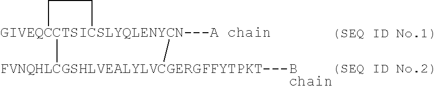
The insulin B chain possesses 2 cysteinyl residues at positions 7 and 19. Both of these are involved in inter-chain disulfides with cysteinyl residues 7 and 20 of the insulin A chain. Cysteinyl residues 6 and 11 of the A chain are involved in an intra-chain disulfide bond. The free insulin B chain peptide or the peptide fragment 9-23 of the insulin B chain both contain sulfhydryl residues that are prone to oxidation which might form disulfide bonds that result in polymerization of the peptides. This makes such peptides with cysteinyl residues less desirable in formulations at or near neutral pH, which is the pH most suitable for pharmaceutical use.

The present invention involves modified human insulin B chain peptides in which either one or both of the cysteinyl residues in the peptide is replaced by substituting serine, threonine, or alanine residues in their place. Such modified insulin B chain peptide analogs should function as immunomimic equivalents of the native insulin B chain, but should not be prone to oxidative polymerization upon storage at neutral pH. Either alanine, serine or threonine substitutions should preserve the hydrodynamic qualities near to those of the native B-chain. The modified peptides of the invention are useful in compositions for the immunotherapeutic treatment of diabetes or pre-diabetic conditions in human subjects.

Immunotherapeutic treatment of human patients with the insulin B chain or the 9-23 peptide fragment of the insulin B chain are disclosed in U.S. Pat. Nos. 5,891,435 and 5,594,100 and published Patent Application US2003/0045467, the disclosures of which are hereby incorporated by reference in their entirety.

The peptide analogs comprising a single cysteine substitution (i.e. Cys7 and Ser/Thr/Ala19 or Ser/Thr/Ala7 and Cys19) are suitable for preparation of B-chain dimers such as homodisulfide dimers by controlled oxidation. Such modified insulin B chain peptide homodisulfide dimer analogs should function as immunomimic equivalents of the native insulin B chain, but are not prone to further oxidative polymerization upon storage at neutral pH that is detrimental to formulation of a stable pharmaceutical product. Either alanine, serine or threonine substitutions at the residual cysteine site should preserve the hydrodynamic qualities of the peptide near to those of the native B-chain. The modified homodisulfide dimer peptides of the invention are useful in compositions for the immunotherapeutic treatment of diabetes or pre-diabetic conditions in human subjects.

In addition, the peptide analogs comprising a single cysteine substitution (i.e. Cys7 and Ser/Thr/Ala19 or Ser/Thr/Ala7 and Cys19) are also suitable for cross-linking to immunogenic proteinaceous carriers such as Diphtheria toxoid (DT), Keyhole Limpet Hemocyanin (KLH), Tetanus toxoid (TT) or Cholera B toxoid. The immunogenic proteins such as DT and TT carry promiscuous T cell epitopes. The cross-linking of the insulin B chain analogs to immunogenic carriers such as DT or TT may be accomplished using bi-functional cross linking agents suitable for coupling via amino groups on the toxoid molecules and via the sole sulfhydryl group of the respective B chain analogs. Such toxoid conjugated insulin B chain epitopes can be expected to provide for superior immunogenicity in both initial immune response as well as superior maintenance of the immune response compared with the insulin B chain or insulin B chain analogs themselves.

The B chain analogs and their toxoid conjugated derivatives may be formulated with adjuvants such as alum or in incomplete Freund's like emulsions in order to maximize their utility as immunogens.

It has been reported that aqueous formulations of insulin B chain 9-23 can induce an immune response to insulin when administered subcutaneously in NOD mice and can prevent the onset of diabetes in that animal model. Moreover, it has been reported that partial protection against diabetes in NOD mice is obtained after immunization with insulin B chain fragment 9-23 administered intranasally. B Chain fragment 9-23 contains one cysteinyl residue which can be expected to undergo oxidative dimerization in aqueous formulations at neutral or near neutral pH. While, in principle, dimerization might not influence adversely the immunogenic quality of the 9-23 insulin B chain fragment, it would compromise the pharmaceutical acceptability of this molecule in formulations prepared at or near neutral pH values. To avoid having a mixture of monomers and dimers where the insulin B chain peptide analogs contain a cysteine residue, such analogs may be provided in the form of dimers substantially free of the monomer. The substitution of serine, threonine or alanine at the single cysteinyl residue of insulin B chain fragment 9-23 would provide for an immunologically equivalent molecule with substantially the same hydrodynamic qualities as the “native” 9-23 fragment, but without the pharmaceutically detrimental tendency for oxidative dimerization.

Other embodiments of the present invention concern immunogenic compositions of insulin peptide analog dimers. These dimers include homodimers where the dimer is prepared by coupling two identical insulin B chain analog molecules through a disulfide bond on a cysteine residue in each of the peptides or heterodimers where two different insulin B chain analog molecules are coupled through a cysteine to cysteine disulfide bond. For example homodisulfide dimers of the human insulin B chain fragment 9-23 may be produced by controlled oxidation. Such modified insulin B chain 9-23 fragment homodisulfide dimer analogs should function as immunomimic equivalents of the 9-23 B chain fragment monomer, but should not be prone to oxidative polymerization upon storage at neutral pH. Oxidative polymerization would be detrimental to formulation of a stable pharmaceutical product.

Methods for producing the insulin B chain analogs as pharmaceutically acceptable immunogens according to the invention include the following examples:

### EXAMPLE 1

Selection of Immunogen Vehicle

Native B-insulin may be formulated as a 30:70 water-in-oil (w/o) emulsion by homogenization of a 30:70 aqueous/oil pre-mix. The continuous oily phase can be prepared from squalene, squalane, or combinations of squalane and squalene suitably formulated with emulsifiers such as Mannide monooleate (MMO), Polyoxyl-40-hydrogenated castor oil (POCO), or similar amphipathic agents and their mixtures. Alternatively, native B-insulin may be formulated as a 50:50 w/o emulsion by homogenization of a 50:50 aqueous/oil pre-mix based on mineral oil formulated with emulsifiers, for example Mannide monooleate, Polyoxyl-40-hydrogenated castor oil, or similar amphipathic agents and their mixtures. Formulations suitable for use as the continuous oily phase in water-in-oil emulsions are available as Montanide formulations from SEPPIC, Paris, France.

The resultant water-in-oil emulsions can be used to immunize NOD mouse or pre-diabetic rat, or in pre-diabetic or diabetic patients and their immune responses compared.

A water-in-oil emulsion may also be prepared by mixing aqueous native insulin B-chain in phosphate buffered saline (PBS) with a suitably formulated oily vehicle as a 30:70 aqueous/oil pre-mix but without homogenization. The pre-mix is then shaken vigorously by hand or vortexed for about 30 to 120 seconds prior to immunization in NOD mouse or pre-diabetic rat model, or in pre-diabetic or diabetic patients.

In addition, an “active” water-in-oil emulsion may also be provided by homogenizing native insulin B chain in PBS with a suitably formulated oily vehicle pre-mix at a ratio of 30:70 water:oil (w/o) and then adjusting the “active” emulsion to the desired strength by dilution of the “active” emulsion with a similarly prepared “placebo” emulsion. Thereby, the antigen “dose” and the “adjuvant” dose ratio may be modified in order to provide an immunogenic composition which can be optimized to maximize the immunogenic response and minimize the inflammatory reaction at the site of injection.

The formulation component ratios and water:oil ratio of the dispersed (water) and continuous (oil) phases may be further adjusted to optimize the robustness of the formulations for processing and to maximize the shelf life of the emulsions. The stability and pharmaceutical acceptability of the various emulsions may be measured by their water globule size distribution, viscosity, and ability to maintain an emulsion without phase separation over time at sub-zero temperatures, at refrigerated temperatures (2-8° C.), at room temperature (25° C.), and at higher more stressful temperatures (≧40° C.).

### EXAMPLE 2

Alternative Insulin Antigen Formulations:

Insulin B chain analogs suitably modified to eliminate the potential for oxidative polymerization inherently associated with native insulin B chain by substitution of cysteine with either serine, threonine, or alanine can be formulated in the emulsions provided described above. Suitable insulin B chain analogs can be made by any method known to the art and include:

Insulin-Ser(Thr/Ala)7, Ser(Thr/Ala)19 B chain analogs:

| | |
|---|---|
| FVNQHLSGSHLVEALYLVSGERGFFYTPKA | (SEQ ID No. 3) |
| FVNQHLSGSHLVEALYLVTGERGFFYTPKA | (SEQ ID No. 4) |
| FVNQHLSGSHLVEALYLVAGERGFFYTPKA | (SEQ ID No. 5) |
| FVNQHLTGSHLVEALYLVSGERGFFYTPKA | (SEQ ID No. 6) |
| FVNQHLTGSHLVEALYLVTGERGFFYTPKA | (SEQ ID No. 7) |
| FVNQHLTGSHLVEALYLVAGERGFFYTPKA | (SEQ ID No. 8) |
| FVNQHLAGSHLVEALYLVSGERGFFYTPKA | (SEQ ID No. 9) |
| FVNQHLAGSHLVEALYLVTGERGFFYTPKA | (SEQ ID No. 10) |
| FVNQHLAGSHLVEALYLVAGERGFFYTPKA | (SEQ ID No. 11) |
| FVNQHLSGSHLVEALYLVSGERGFFYTPKT | (SEQ ID No. 12) |
| FVNQHLSGSHLVEALYLVTGERGFFYTPKT | (SEQ ID No. 13) |
| FVNQHLSGSHLVEALYLVAGERGFFYTPKT | (SEQ ID No. 14) |
| FVNQHLTGSHLVEALYLVSGERGFFYTPKT | (SEQ ID No. 15) |
| FVNQHLTGSHLVEALYLVTGERGFFYTPKT | (SEQ ID No. 16) |
| FVNQHLTGSHLVEALYLVAGERGFFYTPKT | (SEQ ID No. 17) |
| FVNQHLAGSHLVEALYLVSGERGFFYTPKT | (SEQ ID No. 18) |
| FVNQHLAGSHLVEALYLVTGERGFFYTPKT | (SEQ ID No. 19) |
| FVNQHLAGSHLVEALYLVAGERGFFYTPKT | (SEQ ID No. 20) |
| FVNQHLSGSHLVEALYLVSGERGFFYTPKS | (SEQ ID No. 21) |
| FVNQHLSGSHLVEALYLVTGERGFFYTPKS | (SEQ ID No. 22) |
| FVNQHLSGSHLVEALYLVAGERGFFYTPKS | (SEQ ID No. 23) |
| FVNQHLTGSHLVEALYLVSGERGFFYTPKS | (SEQ ID No. 24) |
| FVNQHLTGSHLVEALYLVTGERGFFYTPKS | (SEQ ID No. 25) |
| FVNQHLTGSHLVEALYLVAGERGFFYTPKS | (SEQ ID No. 26) |
| FVNQHLAGSHLVEALYLVSGERGFFYTPKS | (SEQ ID No. 27) |
| FVNQHLAGSHLVEALYLVTGERGFFYTPKS | (SEQ ID No. 28) |
| FVNQHLAGSHLVEALYLVAGERGFFYTPKS | (SEQ ID No. 29) |

Insulin-Ser(Thr/Ala)7, CySH19 B chain analogs:

| | |
|---|---|
| FVNQHLSGSHLVEALYLVCGERGFFYTPKA | (SEQ ID No. 30) |
| FVNQHLTGSHLVEALYLVCGERGFFYTPKA | (SEQ ID No. 31) |
| FVNQHLAGSHLVEALYLVCGERGFFYTPKA | (SEQ ID No. 32) |
| FVNQHLSGSHLVEALYLVCGERGFFYTPKT | (SEQ ID No. 33) |
| FVNQHLTGSHLVEALYLVCGERGFFYTPKT | (SEQ ID No. 34) |
| FVNQHLAGSHLVEALYLVCGERGFFYTPKT | (SEQ ID No. 35) |
| FVNQHLSGSHLVEALYLVCGERGFFYTPKS | (SEQ ID No. 36) |
| FVNQHLTGSHLVEALYLVCGERGFFYTPKS | (SEQ ID No. 37) |
| FVNQHLAGSHLVEALYLVCGERGFFYTPKS | (SEQ ID No. 38) |

Insulin-CySH7, Ser(Thr/Ala)19 B chain analog

| | |
|---|---|
| FVNQHLCGSHLVEALYLVSGERGFFYTPKA | (SEQ ID No. 39) |
| FVNQHLCGSHLVEALYLVTGERGFFYTPKA | (SEQ ID No. 40) |
| FVNQHLCGSHLVEALYLVAGERGFFYTPKA | (SEQ ID No. 41) |
| FVNQHLCGSHLVEALYLVSGERGFFYTPKT | (SEQ ID No. 42) |
| FVNQHLCGSHLVEALYLVTGERGFFYTPKT | (SEQ ID No. 43) |
| FVNQHLCGSHLVEALYLVAGERGFFYTPKT | (SEQ ID No. 44) |
| FVNQHLCGSHLVEALYLVSGERGFFYTPKS | (SEQ ID No. 45) |
| FVNQHLCGSHLVEALYLVTGERGFFYTPKS | (SEQ ID No. 46) |
| FVNQHLCGSHLVEALYLVAGERGFFYTPKS | (SEQ ID No. 47) |

Insulin 9-23, Ser(Thr/Ala)19 B chain analogs:

| | |
|---|---|
| SHLVEALYLVSGERG | (SEQ ID No. 48) |
| SHLVEALYLVTGERG | (SEQ ID No. 49) |
| SHLVEALYLVAGERG | (SEQ ID No. 50) |

These insulin B chain analogs may be formulated with a pharmaceutically acceptable carrier to be used as therapeutic vaccines or toleragens for the treatment of Type 1 diabetes. The insulin B chain analogs may also be administered with an immunogenic carrier such as alum or a toxoid or directly conjugated to an immunogenic carrier such as a toxoid and formulated in a pharmaceutically acceptable carrier that may optionally contain an acceptable adjuvant. The therapeutic vaccines or tolerogens may be administered using treatment methods described in the art. In certain embodiments a water-in-oil emulsion may be used to formulate pharmaceutically acceptable immunogens for the treatment of diabetes or pre-diabetic conditions in human patients.

### EXAMPLE 3

Preparation of Insulin B Chain Analog-Toxoid Conjugates

High titers of antibodies in initial immunogenicity and a sustained immune response may be obtained by conjugating suitable insulin B chain analogs to appropriate immunogenic carrier proteins possessing promiscuous T cell epitopes. In specific embodiments of the invention immunogenic carriers that primarily elicit a Th 2 type IgG, or mixed Th1/Th2 type immune response in a human patient are used. Suitable protein carriers include Diphtheria toxoid (DT), Tetanus toxoid (TT), Keyhole Limpet Hemocyanin (KLH), or Cholera B toxoid. The conjugation may for example, be accomplished via a sulfide linkage in the case of those insulin B chain analogs having at least one sulfhydryl moiety, or through the amino functions of the analogues. The insulin B chain analogs having at least one sulfhydryl available for conjugation, include for example:

Insulin-Ser(Thr/Ala)7, CySH19 B chain analogs:

| | |
|---|---|
| FVNQHLSGSHLVEALYLVCGERGFFYTPKA | (SEQ ID No. 30) |
| FVNQHLAGSHLVEALYLVCGERGFFYTPKS | (SEQ ID No. 38) |
| FVNQHLTGSHLVEALYLVCGERGFFYTPKA | (SEQ ID No. 31) |
| FVNQHLAGSHLVEALYLVCGERGFFYTPKA | (SEQ ID No. 32) |
| FVNQHLSGSHLVEALYLVCGERGFFYTPKT | (SEQ ID No. 33) |
| FVNQHLTGSHLVEALYLVCGERGFFYTPKT | (SEQ ID No. 34) |
| FVNQHLAGSHLVEALYLVCGERGFFYTPKT | (SEQ ID No. 35) |
| FVNQHLSGSHLVEALYLVCGERGFFYTPKS | (SEQ ID No. 36) |
| FVNQHLTGSHLVEALYLVCGERGFFYTPKS | (SEQ ID No. 37) |
| FVNQHLAGSHLVEALYLVCGERGFFYTPKS | (SEQ ID No. 38) |

Insulin-CySH7, Ser(Thr/Ala)19 B chain analogs:

| | |
|---|---|
| FVNQHLCGSHLVEALYLVSGERGFFYTPKA | (SEQ ID No. 39) |
| FVNQHLCGSHLVEALYLVTGERGFFYTPKA | (SEQ ID No. 40) |
| FVNQHLCGSHLVEALYLVAGERGFFYTPKA | (SEQ ID No. 41) |
| FVNQHLCGSHLVEALYLVSGERGFFYTPKT | (SEQ ID No. 42) |
| FVNQHLCGSHLVEALYLVTGERGFFYTPKT | (SEQ ID No. 43) |
| FVNQHLCGSHLVEALYLVAGERGFFYTPKT | (SEQ ID No. 44) |
| FVNQHLCGSHLVEALYLVSGERGFFYTPKS | (SEQ ID No. 45) |
| FVNQHLCGSHLVEALYLVTGERGFFYTPKS | (SEQ ID No. 46) |
| FVNQHLCGSHLVEALYLVAGERGFFYTPKS | (SEQ ID No. 47) |

Insulin 9-23CySH, Ser(Thr/Ala)19 B chain analogs:

| | |
|---|---|
| SHLVEALYLVSGERC | (SEQ ID No. 51) |
| SHLVEALYLVTGERC | (SEQ ID No. 52) |
| SHLVEALYLVAGERC | (SEQ ID No. 53) |

A. Insulin Cys7-23, Ser(Thr/Ala)19 B chain analogs

| | |
|---|---|
| CGSHLVEALYLVSGERG | (SEQ ID No. 54) |
| CGSHLVEALYLVTGERG | (SEQ ID No. 55) |
| CGSHLVEALYLVAGERG | (SEQ ID No. 56) |

In addition insulin B chain analogs selected from SEQ ID Nos. 3-29 are also suitable for coupling to a carrier protein. In particular, the insulin B chain analogs selected from SEQ ID Nos. 3-29 with a cysteine residue at either the amino terminal or at the carboxy terminal, either with or without an intervening spacer sequence of two to six neutral amino acid residues, are suitable for coupling to carrier proteins.

The immunogenic carrier such as DT or TT can be purified free from extraneous non-specific protein fragments by diafiltration with 0.1 M sodium phosphate buffer, 1 mM in EDTA, pH 6.6±0.2 (PA buffer, pH 6.6) to remove low molecular weight impurities and degradation products. For example in the instance of DT, the purified DT is adjusted to about 20 mg/ml with PA buffer, pH 6.6 and then reacted for 1-3 hours at room temperature with an excess of the bi-functional cross-linker N-succinimidyl-6-maleimdocaproate (EMCS) in 0.1 M sodium phosphate buffer, 1 mM in EDTA, pH 6.6±0.2. For this purpose, EMCS is dissolved in N,N-dimethylformamide (DMF) and added dropwise over a period of 10 minutes to the aqueous toxoid solution. EMCS reacts with free amino groups on the protein toxoid via its active ester moiety, thereby introducing maleimidyl groups available to react with free sulfhydryl groups. Post reaction with EMCS, the “activated” toxoid is purified free from excess reactants by diafiltration with 0.1 M sodium citrate buffer, 1 mM in EDTA, pH 6.0±0.2 (CC buffer, pH 6.0). The respective sulfhydryl-containing insulin B chain analog, dissolved in CC buffer, pH 6.0 is added to the Maleimidyl-toxoid and allowed to react overnight at room temperature. The analog-DT conjugate is purified from excess reactants by diafiltration with phosphate buffered saline, pH 7.2±0.2 (PBS, pH 7.2). In an alternative embodiment the analog peptide may be conjugated to the toxoid through a spacer peptide containing a Cysteine residue by methods known in the art.

In each case, the ratio of peptide analog coupled to the toxoid will be varied in the range of 5 to 25 moles of analog per mole of toxoid. The optimal analog-to-toxoid substitution ratio providing for a pharmaceutically acceptable conjugate (solubility and stability in PBS, pH 7.2) and eliciting an acceptable immune response after formulation in the emulsion may be selected after immunization in NOD mouse or prediabetic rat model.

### EXAMPLE 4

Preparation of Insulin B Chain Homodisulfide Dimer Analogs

Stable immunogenic pharmaceutical compositions may be obtained by forming dimers through the controlled oxidation of Cysteine monosubstituted insulin B chain analogs. Cysteine monosubstituted insulin B chain analogs suitable for the preparation of homodisulfide dimer analogs of Insulin B chain include those peptides comprising a single cysteine residue, e.g., selected from those identified herein as SEQ ID Nos. 30-38, 39-47, and 51-56. In addition, the 9-23 fragment of the native insulin B chain, which has a single cysteine corresponding to position 19 of the insulin B chain, is also suitable:
SHLVEALYLVCGERG (SEQ ID No.57)

In each case, the chosen peptide is dissolved in phosphate buffered saline, pH 7.2±0.2 at room temperature. Pure, filtered oxygen is bubbled through the peptide solution in order to promote oxidative dimerization of the cysteine residues to form their respective homodisulfide dimers. The reaction is periodically monitored by sampling the reaction mixture and analysis for monomer dimer ration by reverse phase high pressure liquid chromatography, or by analysis for residual free sulfhydryl residues by reaction with 5, 5′-dithio-bis-(2-nitrobenzoic acid) (Ellman's reagent).

A dimer linked via the sulfhydryl groups on the cysteine residues thus has a structure, e.g., as depicted below: 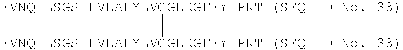
wherein the bond represents a disulfide bond between the sulfhydryl groups of the cysteine residues.

These insulin B chain homodisulfide dimer analogs, optionally containing an acceptable adjuvant may be used as therapeutic vaccines or tolerogens using treatment methods described in the art. In certain embodiments the optimal water-in-oil emulsion may be used to formulate pharmaceutically acceptable immunogens for the treatment of diabetes or pre-diabetic conditions.

### EXAMPLE 5

Preparation of Insulin B Chain Analog Heterodisulfide Dimers

Heterodisulfide dimers of insulin B chain analogs may be produced by the reaction of a monocysteine B chain analog with 5, 5′-dithio-bis-(2-nitrobenzoic acid) (DTNB). This will activate the cysteine residue sulfhydryl group by forming a mixed disulfide with 5-thio-2-nitrobenzoic acid. The DTNB-activated B chain analog is purified from excess DTNB and free 5-thio-2-nitro-benzoate and is then reacted with a second different monocysteine B chain analog to form the heterodisulfide insulin B chain analog with the elimination of 5-thio-2-nitrobenzoic acid. Both the activation reaction and the disulfide formation reaction can be followed by monitoring the elimination of 5-thio-2-nitro-benzoate spectrophotometrically by measuring its absorption at 412 nm. Heterodisulfide insulin B chain analogs may confer a broader immune response in the type 1 diabetic population. This process of specific formation of heterodisulfide dimers may be used to introduce additional substitutions of amino acid residues into any of the available sites in each respective B chain analog so as to produce a diverse range of heterodisulfide dimers of insulin B chain analogs useful as immunotherapeutic agents for tolerization in type 1 diabetes.

1. An immunogenic composition comprising an insulin B chain analog peptide, wherein at least one of the two cysteine residues in the insulin B chain is substituted by a serine, threonine or alanine residue.

2. An immunogenic composition according to claim 1 wherein in the insulin B chain analog peptide, the threonine residue at the carboxy terminal position of the insulin B chain is substituted by an alanine residue.

3. An immunogenic composition according to claim 1 wherein in the insulin B chain analog peptide, the threonine residue at the carboxy terminal position of the insulin B chain is substituted by a serine residue.

4. An immunogenic composition according to claim 1 wherein in the insulin B chain analog peptide, the residue at the carboxy terminal is a threonine residue.

5. An immunogenic composition according to claim 1 wherein the insulin B chain analog is a peptide selected from the group consisting of SEQ ID Nos. 3-56.

6. An immunogenic composition according to claim 1 wherein the insulin B chain analog is a peptide selected from the group consisting of SEQ ID Nos. 3-11, 30-32, 39-41, 48-50, and 51-56.

7. An immunogenic composition according to claim 1 wherein the insulin B chain analog peptide is conjugated to an immunogenic carrier.

8. An immunogenic composition according to claim 2 wherein the insulin B chain analog peptide is conjugated to an immunogenic carrier.

9. An insulin B chain analog peptide dimer comprising a first insulin B chain analog peptide wherein one of the two cysteine residues in the insulin B chain has been substituted or deleted and the remaining cysteine is bound to the cysteine of a second insulin B chain analog peptide by a disulfide bond.

10. An insulin B chain analog peptide dimer according to claim 9 comprising a first insulin B chain analog peptide wherein one of the two cysteine residues in the insulin B chain analog peptide is substituted by a serine, a threonine or alanine residue and the remaining cysteine is bound to the cysteine of a second insulin B chain analog peptide by a disulfide bond.

11. An insulin B chain analog peptide dimer according to claim 9 comprising a first insulin B chain analog peptide wherein a portion of the insulin B chain comprising one of the two cysteine residues in the insulin B chain is deleted and the remaining cysteine is bound to the cysteine of a second insulin B chain molecule by a disulfide bond.

12. An insulin B chain analog peptide dimer according to claim 1 wherein the first insulin B chain peptide is of SEQ ID No. 57.

13. An insulin B chain analog peptide dimer according to claim 11 wherein the first and second insulin B chain analog peptides are of SEQ ID No. 57.

14. The insulin B chain analog peptide dimer of claim 10 wherein the first insulin B chain analog peptide is selected from the group consisting of SEQ ID Nos. 30-38, 39-47, and 51-56.

15. The insulin B chain analog peptide dimer of claim 14 wherein the first insulin B chain peptide analog is selected from the group consisting of SEQ ID Nos. 30-32, 39-41, and 51-56.

16. The insulin B chain analog peptide dimer of claim 14 wherein the first insulin B chain peptide analog is of SEQ ID 33.

17. The insulin B chain analog peptide dimer of claim 16 wherein the second insulin B chain peptide is of SEQ ID 33.

18. The insulin B chain analog peptide dimer of claim 9 wherein the first and second insulin B chain peptides are the same.

19. An immunogenic composition comprising a first insulin B chain analog peptide wherein one of the two cysteine residues in the insulin B chain has been substituted or deleted and the remaining cysteine is bound to the cysteine of a second insulin B chain analog peptide by a disulfide bond.

20. An immunogenic composition according to claim 1 wherein the insulin B chain analog peptide is in the form of an insulin B chain analog peptide dimer comprising a first insulin B chain peptide analog wherein one of the two cysteine residues in the insulin B chain peptide analog is substituted by a serine, a threonine or alanine residue and the remaining cysteine is bound to the cysteine of a second insulin B chain molecule by a disulfide bond.

21. An immunogenic composition according to claim 19 wherein the first and second insulin B chain analog peptides are the same.

22. An immunogenic composition according to claim 20 wherein the first and second insulin B chain analog peptides are of SEQ ID 33.

23. An immunogenic composition according to claim 1 in the form of a water-in-oil emulsion.

24. An immunogenic composition according to claim 23 comprising an emulsifier selected from mannide monooleate and polyoxyl-40-hydrogenated castor oil.

25. An immunogenic composition according to claim 21 wherein the oil phase of the water-in-oil emulsion comprises squalene, squalane, or mixtures thereof.

26. An immunogenic composition according to claim 21 comprising (i) mannide monooleate and (ii) an oil phase comprising squalene, squalane, or mixtures thereof.

27. A method of treating diabetes or a pre-diabetic condition in a human subject comprising administering to the subject a composition of claim 1.

28. A method of inducing immunological tolerance to insulin in a patient having autoantibodies to insulin comprising administering to the patient an immunologically tolerizing amount of a composition of claim 1.


SEQUENCE LISTING (57 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 30 aa, Artificial Sequence
  Insulin B chain analog
FVNQHLSGSHLVEALYLVSGERGFFYTPKA

SEQ ID NO: 4 - protein, 30 aa, Artificial Sequence
  Insulin B chain analog
FVNQHLSGSHLVEALYLVTGERGFFYTPKA

SEQ ID NO: 5 - protein, 30 aa, Artificial Sequence
  Insulin B chain analog
FVNQHLSGSHLVEALYLVAGERGFFYTPKA

SEQ ID NO: 6 - protein, 30 aa, Artificial Sequence
  Insulin B chain analog
FVNQHLTGSHLVEALYLVSGERGFFYTPKA

SEQ ID NO: 7 - protein, 30 aa, Artificial Sequence
  Insulin B chain analog
FVNQHLTGSHLVEALYLVTGERGFFYTPKA

SEQ ID NO: 8 - protein, 30 aa, Artificial Sequence
  Insulin B chain analog
FVNQHLTGSHLVEALYLVAGERGFFYTPKA

SEQ ID NO: 9 - protein, 30 aa, Artificial Sequence
  Insulin B chain analog
FVNQHLAGSHLVEALYLVSGERGFFYTPKA

SEQ ID NO: 10 - protein, 30 aa, Artificial Sequence
  Insulin B chain analog
FVNQHLAGSHLVEALYLVTGERGFFYTPKA

SEQ ID NO: 11 - protein, 30 aa, Artificial Sequence
  Insulin B chain analog
FVNQHLAGSHLVEALYLVAGERGFFYTPKA

SEQ ID NO: 12 - protein, 30 aa, Artificial Sequence
  Insulin B chain analog
FVNQHLSGSHLVEALYLVSGERGFFYTPKT

SEQ ID NO: 13 - protein, 30 aa, Artificial Sequence
  Insulin B chain analog
FVNQHLSGSHLVEALYLVTGERGFFYTPKT

SEQ ID NO: 14 - protein, 30 aa, Artificial Sequence
  Insulin B chain analog
FVNQHLSGSHLVEALYLVAGERGFFYTPKT

SEQ ID NO: 15 - protein, 30 aa, Artificial Sequence
  Insulin B chain analog
FVNQHLTGSHLVEALYLVSGERGFFYTPKT

SEQ ID NO: 16 - protein, 30 aa, Artificial Sequence
  Insulin B chain analog
FVNQHLTGSHLVEALYLVTGERGFFYTPKT

SEQ ID NO: 17 - protein, 30 aa, Artificial Sequence
  Insulin B chain analog
FVNQHLTGSHLVEALYLVAGERGFFYTPKT

SEQ ID NO: 18 - protein, 30 aa, Artificial Sequence
  Insulin B chain analog
FVNQHLAGSHLVEALYLVSGERGFFYTPKT

SEQ ID NO: 19 - protein, 30 aa, Artificial Sequence
  Insulin B chain analog
FVNQHLAGSHLVEALYLVTGERGFFYTPKT

SEQ ID NO: 20 - protein, 30 aa, Artificial Sequence
  Insulin B chain analog
FVNQHLAGSHLVEALYLVAGERGFFYTPKT

SEQ ID NO: 21 - protein, 30 aa, Artificial Sequence
  Insulin B chain analog
FVNQHLSGSHLVEALYLVSGERGFFYTPKS

SEQ ID NO: 22 - protein, 30 aa, Artificial Sequence
  Insulin B chain analog
FVNQHLSGSHLVEALYLVTGERGFFYTPKS

SEQ ID NO: 23 - protein, 30 aa, Artificial Sequence
  Insulin B chain analog
FVNQHLSGSHLVEALYLVAGERGFFYTPKS

SEQ ID NO: 24 - protein, 30 aa, Artificial Sequence
  Insulin B chain analog
FVNQHLTGSHLVEALYLVSGERGFFYTPKS

SEQ ID NO: 25 - protein, 30 aa, Artificial Sequence
  Insulin B chain analog
FVNQHLTGSHLVEALYLVTGERGFFYTPKS

SEQ ID NO: 26 - protein, 30 aa, Artificial Sequence
  Insulin B chain analog
FVNQHLTGSHLVEALYLVAGERGFFYTPKS

SEQ ID NO: 27 - protein, 30 aa, Artificial Sequence
  Insulin B chain analog
FVNQHLAGSHLVEALYLVSGERGFFYTPKS

SEQ ID NO: 28 - protein, 30 aa, Artificial Sequence
  Insulin B chain analog
FVNQHLAGSHLVEALYLVTGERGFFYTPKS

SEQ ID NO: 29 - protein, 30 aa, Artificial Sequence
  Insulin B chain analog
FVNQHLAGSHLVEALYLVAGERGFFYTPKS

SEQ ID NO: 30 - protein, 30 aa, Artificial Sequence
  Insulin B chain analog
FVNQHLSGSHLVEALYLVCGERGFFYTPKA

SEQ ID NO: 31 - protein, 30 aa, Artificial Sequence
  Insulin B chain analog
FVNQHLTGSHLVEALYLVCGERGFFYTPKA

SEQ ID NO: 32 - protein, 30 aa, Artificial Sequence
  Insulin B chain analog
FVNQHLAGSHLVEALYLVCGERGFFYTPKA

SEQ ID NO: 33 - protein, 30 aa, Artificial Sequence
  Insulin B chain analog
FVNQHLSGSHLVEALYLVCGERGFFYTPKT

SEQ ID NO: 34 - protein, 30 aa, Artificial Sequence
  Insulin B chain analog
FVNQHLTGSHLVEALYLVCGERGFFYTPKT

SEQ ID NO: 35 - protein, 30 aa, Artificial Sequence
  Insulin B chain analog
FVNQHLAGSHLVEALYLVCGERGFFYTPKT

SEQ ID NO: 36 - protein, 30 aa, Artificial Sequence
  Insulin B chain analog
FVNQHLSGSHLVEALYLVCGERGFFYTPKS

SEQ ID NO: 37 - protein, 30 aa, Artificial Sequence
  Insulin B chain analog
FVNQHLTGSHLVEALYLVCGERGFFYTPKS

SEQ ID NO: 38 - protein, 30 aa, Artificial Sequence
  Insulin B chain analog
FVNQHLAGSHLVEALYLVCGERGFFYTPKS

SEQ ID NO: 39 - protein, 30 aa, Artificial Sequence
  Insulin B chain analog
FVNQHLCGSHLVEALYLVSGERGFFYTPKA

SEQ ID NO: 40 - protein, 30 aa, Artificial Sequence
  Insulin B chain analog
FVNQHLCGSHLVEALYLVTGERGFFYTPKA

SEQ ID NO: 41 - protein, 30 aa, Artificial Sequence
  Insulin B chain analog
FVNQHLCGSHLVEALYLVAGERGFFYTPKA

SEQ ID NO: 42 - protein, 30 aa, Artificial Sequence
  Insulin B chain analog
FVNQHLCGSHLVEALYLVSGERGFFYTPKT

SEQ ID NO: 43 - protein, 30 aa, Artificial Sequence
  Insulin B chain analog
FVNQHLCGSHLVEALYLVTGERGFFYTPKT

SEQ ID NO: 44 - protein, 30 aa, Artificial Sequence
  Insulin B chain analog
FVNQHLCGSHLVEALYLVAGERGFFYTPKT

SEQ ID NO: 45 - protein, 30 aa, Artificial Sequence
  Insulin B chain analog
FVNQHLCGSHLVEALYLVSGERGFFYTPKS

SEQ ID NO: 46 - protein, 30 aa, Artificial Sequence
  Insulin B chain analog
FVNQHLCGSHLVEALYLVTGERGFFYTPKS

SEQ ID NO: 47 - protein, 30 aa, Artificial Sequence
  Insulin B chain analog
FVNQHLCGSHLVEALYLVAGERGFFYTPKS

SEQ ID NO: 48 - protein, 15 aa, Artificial Sequence
  Insulin B chain analog
SHLVEALYLVSGERG

SEQ ID NO: 49 - protein, 15 aa, Artificial Sequence
  Insulin B chain analog
SHLVEALYLVTGERG

SEQ ID NO: 50 - protein, 15 aa, Artificial Sequence
  Insulin B chain analog
SHLVEALYLVAGERG

SEQ ID NO: 51 - protein, 15 aa, Artificial Sequence
  Insulin B chain analog
SHLVEALYLVSGERC

SEQ ID NO: 52 - protein, 15 aa, Artificial Sequence
  Insulin B chain analog
SHLVEALYLVTGERC

SEQ ID NO: 53 - protein, 15 aa, Artificial Sequence
  Insulin B chain analog
SHLVEALYLVAGERC

SEQ ID NO: 54 - protein, 17 aa, Artificial Sequence
  Insulin B chain analog
CGSHLVEALYLVSGERG

SEQ ID NO: 55 - protein, 17 aa, Artificial Sequence
  Insulin B chain analog
CGSHLVEALYLVTGERG

SEQ ID NO: 56 - protein, 17 aa, Artificial Sequence
  Insulin B chain analog
CGSHLVEALYLVAGERG

SEQ ID NO: 57 - protein, 15 aa, Artificial Sequence
  Insulin B chain analog
SHLVEALYLVCGERG